(19) United States
(12) Patent Application Publication
(10) Pub. No.: US 2013/0172271 A1
(43) Pub. Date: Jul. 4, 2013

(54) Pharmaceutical Spray Drying

(71) Applicant: Cynthia Fragale, Kenosha, WI (US)

(72) Inventor: Cynthia Fragale, Kenosha, WI (US)

(21) Appl. No.: 13/734,329

(22) Filed: Jan. 4, 2013

Related U.S. Application Data
(60) Provisional application No. 61/583,083, filed on Jan. 4, 2012.

(51) Int. Cl. A61K 38/12; A61K 31/7052

(52) U.S. Cl. CPC A61K 38/12; A61K 31/7052
USPC 514/21.1; 514/29

(57) ABSTRACT

Methods and formulations for preparing spray dried pharmaceutical products including daptomycin, azithromycin, cyclophosphamide, and voriconazole.

### CROSS-REFERENCE TO RELATED APPLICATION

This application claims the benefit of U.S. provisional application Ser. No. 61/583,083 filed Jan. 4, 2012.

### FIELD

The application relates generally to spray drying pharmaceuticals, and particularly to a method for preparing spray dried vancomycin, daptomycin, azithromycin, cyclophosphamide, and voriconazole.

### BACKGROUND

Vancomycin HCl, daptomycin, azithromycin, cyclophosphamide, and voriconazole are pharmaceutical substances that are usually provided in powdered forms to a health care provider. For example, Vancomycin HCl for Injection (Hospira, Inc., Lake Forest, Ill.) is a sterile dry mixture of vancomycin HCl and may contain a suitable stabilizing agent. These products are generally prepared into their powdered form by lyophilizing (freeze-drying) an aseptically filled solution. The lyophilization process can be inefficient and require expensive production equipment and processing steps.

As an alternative to lyophilization, spray drying involves transformation of a formulation from a fluid state into a dried form by spraying the formulation into a hot drying medium. The formulation can be either a solution, suspension, or a paste. The spray dried product is typically in the form of a powder consisting of single particles or agglomerates, depending upon the physical and chemical properties of the formulation and the dryer design and operation. The basic technique includes the following four steps: a) atomization of the formulation solution into a spray; b) spray-gas contact; c) drying of the spray; and d) separation of the dried product from the drying gas. An aseptic spray drying method can be a more efficient and cost effective process of powder generation compared to lyophilization.

Accordingly, the inventors have identified a need in the art for a suitable formulation and process for spray drying vancomycin, daptomycin, azithromycin, cyclophosphamide, and voriconazole.

### SUMMARY

In one aspect, the invention is directed to a method for preparing a powdered daptomycin, azithromycin, cyclophosphamide, or voriconazole pharmaceutical formulation for injection. The method includes providing a solution comprising daptomycin, azithromycin, cyclophosphamide, or voriconazole and polyethylene glycol (PEG). The solution is spray dried to form a powder that can be reconstituted and administered by IV infusion. In one embodiment, the solution further includes mannitol. In various aspects, the solutions may include about 1-5% PEG and about 1-7% by weight of mannitol. The PEG may be PEG-400. The pH of the solution may be about 4.0-5.5 or about 6.0-7.0.

In another aspect, the invention is directed to a method for preparing a powdered azithromycin formulation for injection. The method includes providing a solution comprising azithromycin and citric acid, and spray drying the solution to form a powder that can be reconstituted and administered by IV infusion. In one aspect, the solution includes about 5-10% azithromycin by weight and about 4-8% citric acid by weight. In other aspect, the solution may further include about 2-8% mannitol and/or about 2-8% PEG-400.

In a further aspect, the invention is directed to a method for preparing a powdered daptomycin formulation for injection. The method includes providing a solution comprising daptomycin and citric acid, and spray drying the solution to form a powder that can be reconstituted and administered by IV infusion. In various aspects, the solution can include about 5-20% daptomycin, about 1-10% mannitol by weight and about 1-10% PEG-400 by weight. For example, the solution may include about 10-12% daptomycin by weight, about 3-5% mannitol by weight, and/or about 3% PEG-400 by weight.

### BRIEF DESCRIPTION OF THE FIGURES

An exemplary embodiment of the present application is described herein with reference to the figures, in which:

FIG. 1 shows a schematic view of an exemplary spray drying system according to an embodiment of the invention;

FIGS. 2a-e show a schematic view of a valve for use in a method according to the invention; and

FIG. 3 is a schematic diagram of a powder sampling system for use in a method according to the invention.

### DESCRIPTION

The invention provides methods and formulations for providing a spray dried vancomycin, daptomycin, azithromycin, cyclophosphamide, or voriconazole product that can be reconstituted and administered by IV infusion. Spray dried pharmaceutical formulations are preferably produced aseptically to avoid the need for further sterilization. The pharmaceutical solution to be spray dried is preferably selected so as to provide (upon spray drying) a substantially uniform powder with a favorable moisture content and reconstitution profile.

In one embodiment, the invention is directed to a formulation including vancomycin HCl, daptomycin, azithromycin, cyclophosphamide, or voriconazole and one or more excipients that are desirable to provide a spray dried formulation suitable for packaging, reconstitution and delivery by IV infusion to a patient. In various embodiments, the formulations suitable for spray drying include vancomycin HCl, daptomycin, azithromycin, cyclophosphamide, or voriconazole and polyethylene glycol (PEG) in a polar solvent, such as water for injection (WFI).

In one particular embodiment, a solution suitable for spray drying includes vancomycin HCl at a concentration of 10-20% by weight, PEG at a concentration of 1-5% by weight and mannitol at a concentration of 1-5% by weight in water for injection. In another embodiment, the solution includes vancomycin HCl (10-20% by weight) and PEG at 1-5% by weight in a citrate buffer. In yet another embodiment, the solution includes vancomycin HCl (10-20% by weight), PEG (1-5% by weight) and ethanol (up to 25% by weight) in water for injection.

Vancomycin HCl API is supplied as a powdered substance with a chromatographic purity of not less than 85%. Daptomycin, Azithromycin, Cyclophosphamide and Voriconazole are also supplied as powdered substances with pharmaceutically acceptable purity. Polyethylene glycol (PEG) and mannitol are available from a number of known suppliers. The PEG and mannitol used in connection with the present invention are preferably of pharmaceutical quality, parenteral grade, and compliant with US Pharmacopeia, Japanese Pharmacopeia, and/or European Pharmacopeia standards. The polyethylene glycol used in accordance with the invention preferably has molecular weight of approximately 380-420 Daltons, and is generally referred to as PEG-400.

In another embodiment, the solution for spray drying vancomycin includes vancomycin HCl at a concentration of about 10% to about 20% (by weight), and more particularly, of about 13% to about 17%. In one embodiment, the solution for spray drying contains approximately 15% (by weight) vancomycin HCl. The solution further includes mannitol and PEG-400 at equal or non-equal concentrations of between about 1% to about 5% (by weight), and more particularly between about 2% and about 4% (by weight). In one embodiment, the solution includes mannitol and PEG-400 in equal concentration of about 3% (by weight) in water for injection (15% vancomycin HCl, 3% PEG-400, 3% mannitol, and 79% water for injection).

In another embodiment, the solution includes PEG-400 and mannitol in equal concentration of 2.5% (by weight). For this specific formulation, the quantitative composition in solution is provided in Table 1 and the quantitative composition of the dried powder is provided in Table 2.

TABLE 1

| Component | Quantity per liter | |
|---|---|---|
| Vancomycin HCl | 150 | g |
| PEG-400 | 25 | g |
| Mannitol | 25 | g |
| HCl | as required to pH 3.4 to 3.6 | |
| NaOH | as required to pH 3.4 to 3.6 | |
| Water for Injection | as required to 1.00 | L |
| Total Volume | 1.00 | L |

TABLE 2

| Component | Quantity per gram |
|---|---|
| Vancomycin HCl | 0.75 g |
| PEG-400 | 0.125 g |
| Mannitol | 0.125 g |
| Total Weight | 1.0 g |

In another embodiment, the solution for spray drying daptomycin includes daptomycin at a concentration of about 5-20% (by weight), and more particularly, of about 7% to about 15%. In one embodiment, the solution for spray drying contains approximately 10% to 12% (by weight) daptomycin. The solution further includes PEG-400 and mannitol at concentrations of between about 1% to about 10% (by weight), and more particularly between about 2% and about 8% (by weight). In one embodiment, the solution includes PEG-400 and mannitol in equal concentration of about 3% (by weight) in water for injection (10% daptomycin, 3% PEG-400, 3% mannitol, and 84% water for injection). In another embodiment, the solution includes daptomycin at about 12% (by weight) with PEG-400 (3% by weight) and mannitol (5% by weight) in WFI.

In another embodiment, a solution for spray drying azithromycin includes azithromycin at a concentration of about 2-25% (by weight), and more particularly about 5% to about 10% by weight, and even more particularly about 6-8% by weight. The solution may further include about 4-8% citric acid by weight. In one embodiment, the solution for spray drying contains approximately 7.2% (by weight) azithromycin and 5.6% (by weight) citric acid in water. The solution may further include PEG-400 and/or mannitol at equal or non-equal concentrations of between about 1% to about 7% (by weight), and more particularly between about 2% and about 7% (by weight). In one embodiment, the solution includes PEG-400 and mannitol in equal concentration of about 2.3% (by weight) in water for injection (7.0% azithromycin, 5.5% citric acid, 2.3% PEG-400, 2.3% mannitol, and 82.9% water for injection).

In various embodiments of the invention, hydrochloric acid, sodium hydroxide, or other known agents can be used to adjust the pH of the vancomycin solution to pH of about 3.0-3.7, for instance about 3.3-3.6, for example about 3.5. In other embodiments of the invention, hydrochloric acid, sodium hydroxide, or other known agents can be used to adjust the pH of the daptomycin solution to about 4.0-5.5, for instance about 4.5-5.0. In another embodiment of the invention, hydrochloric acid, sodium hydroxide, or other known agents can be used to adjust the pH of the azithromycin solution to about 6.0-7.0, for instance about 6.4 to 6.6, for example about 6.5.

In various alternative embodiments, the solution includes vancomycin HCl and PEG-400 in an aqueous citrate buffer (e.g., citric acid/sodium citrate, pH 3.0-3.5). The solution includes vancomycin HCl at a concentration of about 10% to about 20% (by weight), and more particularly, of about 13% to about 17% (by weight). In one embodiment, the solution for spray drying contains approximately 15% (by weight) vancomycin HCl. The solution further includes PEG-400 between about 1% to about 5% (by weight), and more particularly between about 2% to about 4% (by weight). For example, the solution can include PEG-400 at a concentration of approximately 3% (by weight) or at a concentration of approximately 2.5% (by weight). Upon formulation, the pH of the solution can be adjusted as necessary to provide the desired pH.

Other suitable excipients for preparing alternative solutions suitable for spray drying include glycine (3%-7% (by weight)), sucrose (3%-7% (by weight)), trehelose (1%-4% (by weight)), and poloxamer (0.3%-1% (by weight)).

In one particular embodiment, the solution includes cyclophosphamide at a concentration of about 0.1 to about 25% by weight, PEG at a concentration of 1-10% by weight and mannitol at a concentration of 1-10% by weight in water for injection. In another embodiment, the solution includes cyclophosphamide (0.1% to about 25% by weight) and PEG at 1-5% by weight in a citrate buffer. In yet another embodiment, the solution includes cyclophosphamide (about 0.1% to about 25% by weight), PEG (1-5% by weight) and ethanol (up to 25% by weight) in water for injection. The solution may further include PEG-400 and mannitol at concentrations of between about 2% and about 8% (by weight). In one embodiment, the solution includes PEG-400 and mannitol in equal concentration of about 3% (by weight) in water for injection (0.1-25% cyclophosphamide, 3% PEG-400, 3% mannitol, and water for injection). In another embodiment, the solution includes cyclophosphamide at about 10-12% (by weight), PEG-400 (3% by weight) and mannitol (5% by weight) in WFI.

In one particular embodiment, the solution includes voriconazole at a concentration of about 0.1% to about 25% by weight, PEG at a concentration of 1-10% by weight and mannitol at a concentration of 1-10% by weight in water for injection. In another embodiment, the solution includes voriconazole (about 0.1 to about 25% by weight) and PEG at 1-5% by weight in a citrate buffer. In yet another embodiment, the solution includes voriconazole (0.1-25% by weight), PEG (1-5% by weight) and ethanol (up to 25% by weight) in water for injection. The solution further includes PEG-400 and mannitol at concentrations of between about 2% and about 8% (by weight). In one embodiment, the solution includes PEG-400 and mannitol in equal concentration of about 3% (by weight) in water for injection (0.1-25% voriconazole, 3% PEG-400, 3% mannitol, and water for injection). In another embodiment, the solution includes voriconazole at about 10-12% (by weight) with PEG-400 (3% by weight) and mannitol (5% by weight) in WFI.

The spray drying process provides a benefit over lyophilizing pharmaceutical solutions for compounds such as cyclophosphamide, which is a crystalline monohydrate and is soluble in water. Cyclophosphamide monohydrate liquefies when its water of crystallization is lost. Lyophilization of this compound is technically challenging to the extent that the residual moisture content of the cake must be maintained at about 4%. Accordingly, a re-humidification step has been introduced at the end of the lyophilization cycle. Re-humidification requires modification to the existing lyophilization chambers and processes.

Spray drying allows of cyclophosphamide with or without excipients can provide for the proper humidity in the final product by manipulating one or more of the spray drying process parameters such as aspirator speed, humidity of the drying gas, feed rate, and inlet temperature, either separately or in combination (see, e.g., Table 5, below).

The process for spray drying a pharmaceutical formulation of vancomycin HCl, daptomycin, azithromycin, cyclophosphamide, or voriconazole is preferably undertaken aseptically. Prior to the spray drying step, a solution compounding system may be used to rapidly mix the active pharmaceutical ingredient (API), i.e., vancomycin HCl, and excipients in an environmentally controlled system. The system can be a conventional tank and mixer blending system and should minimize operator exposure to the API, and minimize the API's exposure to air/oxygen during the handling, mixing and holding of ingredients prior to spray drying in order to avoid premature degradation of the API if susceptible to oxidation. The compounded solution undergoes a sterile filtration process prior to the aseptic spray drying process. Following the spray drying process, the resulting powder is harvested into sterile vessels. At the appropriate time, dried powder is then transferred to a filler that fills the resulting spray dried vancomycin in pharmaceutical vials, which are then capped and sealed.

The process may utilize pre-cooled, nitrogen-purged WFI that is added to a nitrogen purged tank through a flow meter to measure the quantity of water. Since the process may be weight based, the full quantity of water, which will be based on the weight of the API to be mixed, can be charged initially.

Once compounding activities have been completed and the mixed solution has met the pre-defined chemistry limits (e.g., pH), the solution is transferred to the spray dryer. The actual spray drying involves the atomization of a liquid solution (feedstock) into a spray of droplets, and contacting the droplets with hot gas in a drying chamber. The droplets can be produced by, for example, nozzle atomizers. Evaporation of moisture from the produced droplets and formation of dry particles proceed under controlled temperature and gas flow conditions. When the droplets are small enough and the chamber large enough, the droplets dry before they reach the wall of the chamber. The resulting product is collected as a free-flowing material. Powder is discharged continuously from the drying chamber. Operating conditions and spray dryer design are selected according to the drying characteristics of the product and powder specification.

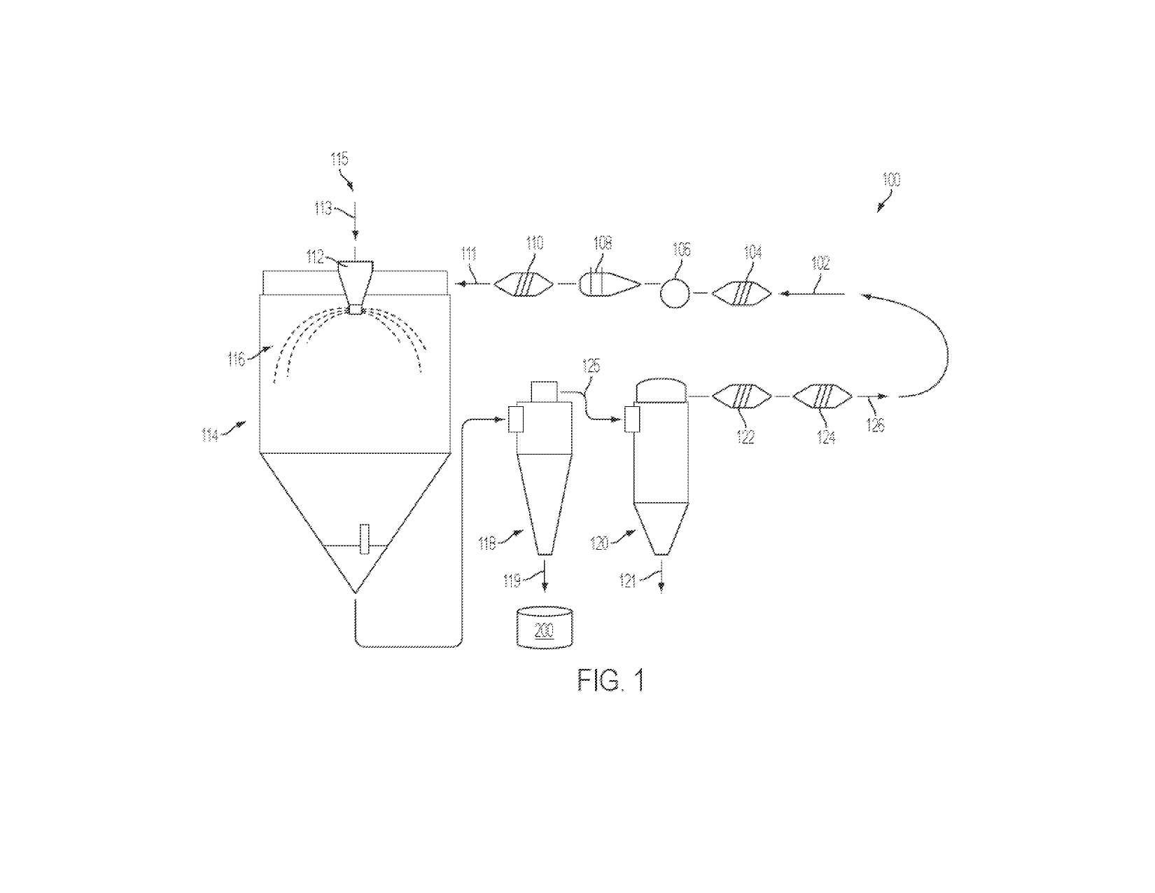

Spray dryers generally include a feedstock pump, an atomizer, a gas heater, a gas disperser, a drying chamber, and systems for exhaust gas cleaning and powder recovery. Referring to FIG. 1, an example spray drying system 100 includes drying gas 102 introduced into a pre-filter 104. In one aspect, the drying gas is nitrogen, and avoids the presence of oxygen. In one embodiment, the drying gas comprises nitrogen with less than 1% oxygen. The drying gas 102 then passes through a fan 106 and a heater 108, which may be an electric heater. The drying gas 102 then passes through a sterilizing gas filter 110 and an inlet gas temperature gauge 111 monitors the inlet gas temperature before it is introduced into a drying chamber 114 via a ceiling gas dispenser. Redundant filtration may be employed to ensure product quality.

The formulation, from the tank and mixer blending system 115, undergoes a sterile filtration process 113 prior to being fed into the drying chamber 114 and atomized by an atomizer 112. The atomizer 112 may be any type of known atomizer that allows for aseptic processing such as a pressure nozzle or a two-fluid nozzle (e.g., available from GEA Process Engineering Inc., Columbia, Md., formerly known as Niro Inc.). The atomizer 112 disperses the liquid formulation into a controlled drop size spray 116. In one particular embodiment, the atomizer is operated with a nozzle protection of 80 kg/hour nitrogen gas at 80° C. The spray 116 is then heated in the drying chamber 114. The heated drying gas evaporates the liquid from the spray and forms dry particles.

After the solution has been atomized and heated, the dry particles exit the drying chamber 116 and proceed into a cyclone 118, which separates the powder from the gas. The powder flows out of the cyclone 118 at outlet 119 into a sterile powder collection vessel 200 and the rest of the gas flows out past an outlet gas temperature gauge 125 toward a cartridge filtration system 120. The cartridge filtration system 120 removes fine particles at outlet 121. The remaining dried gas then flows through a second filter 122 (e.g. a sterile filter), and in some embodiments through a third filter 124, and then back into the drying gas supply at 102. In one embodiment, a vortex eliminator may be used near the bottom of the cyclone 118 to eliminate hot gas from passing through the outlet 119.

As shown in FIG. 1, the dried powder from outlet 119 is collected in a sterile powder collection vessel 200. The collection vessel 200 can be, for example, a 316L grade stainless steel pressure rated vessel, and may be steam sterilized prior to use. The collection vessel 200 may be automatically cleaned, sterilized, and charged with a positive pressure of sterile filtered nitrogen gas.

In an embodiment of the invention shown in FIG. 1, the collection vessel 200 includes a valve assembly 300, which may be a split butterfly valve or a containment valve, that allows for the sterile transfer of the powder from the cyclone 118 of the spray dryer 100 to the collection vessel 200, and then from the collection vessel 200 to an aseptic powder filler (not shown). The valve assembly 300 is located, for example, between the collection vessel 200 and a bottom of the cyclone 118.

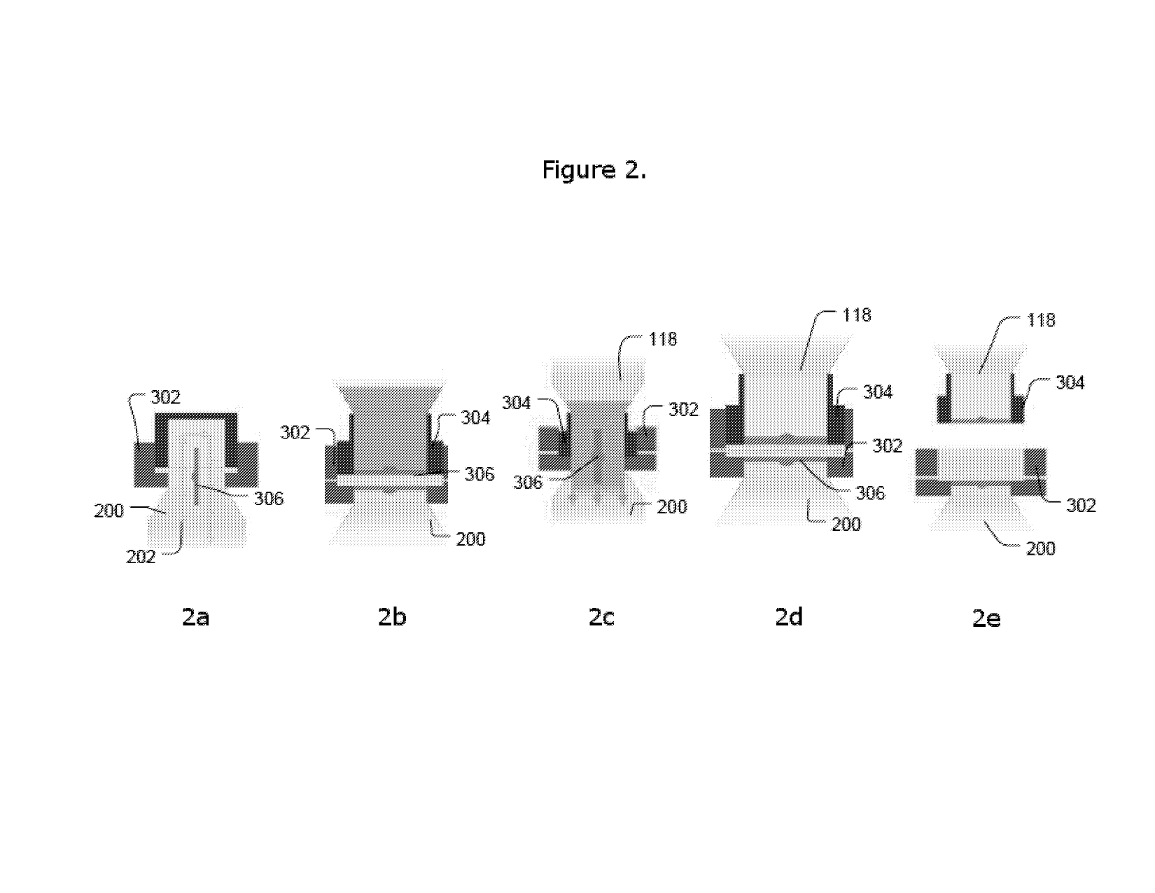

As shown schematically as an exemplary embodiment in FIGS. 2a-e, the valve assembly 300 has an active portion 302 secured to the collection vessel 200 and a passive portion 304 secured to the cyclone 118 of the spray dryer 100. The valve assembly 300 further includes a safety interlock system (not shown) which prevents the active portion 302 from being separated from the passive portion 304 while the valve assembly 300 is open. In one particular embodiment, the valve is a CHARGEPOINT® Excel valve (ChargePoint Technology, Bayville, N.J.).

In an exemplary use, the collection vessel 200 is placed on a small lift mechanism that centers the collection vessel 200 below the spray dryer cyclone 118 for docking During docking, the active portion 302 of the valve assembly 300 is be sterilized in place (SIP) with vaporized hydrogen peroxide (VHP) 202, as shown in FIG. 2a. Next, as shown in FIG. 2b, the passive portion 304 is connected to the active portion 302 and is purged with VHP to decontaminate previously exposed faces of the active and passive portions 302, 304. The valve assembly 300 includes a valve disc 306 that opens to allow transfer of product, or powder, from the spray dryer cyclone 118 into the collection vessel 200, as shown in FIG. 2c.

When the powder transfer is complete, the valve closes and the feedstock is switched from the pharmaceutical formulation to WFI. In an exemplary embodiment using the CHARGEPOINT® Excel valve, the valve is closed and a control system signals the valve actuator to unlock the two sections of the split butterfly valve, as shown in FIG. 2d, thereby allowing collection vessel 200 to be separated from the passive portion 304, as shown in FIG. 2e. An operator may lower the collection vessel 200 via a local control panel and manually place a pressure cap (not shown) on the collection vessel 200 and lock the pressure cap into position. After the pressure cap is in place, the operator may induce the control system via the local control panel to take a pressure reading from the collection vessel and record all salient vessel characteristics (i.e., vessel I.D number, date, time, and collection vessel pressure). When complete, the collection vessel may be stored for later use at a powder filler.

In one embodiment, the spray dryer operates at several times the normal operating pressure of conventional spray dryers, including those considered “pharmaceutical grade”. In particular, the invention operates at about 1.5-7.0 psig (pounds per square inch, gauge), more particularly about 5.5 psig. This higher pressure is required so that the powder transfer vessel is at this higher pressure when powder transfer is complete and the split butterfly valve is closed. This higher pressure allows for pressure reduction as the vessel and powder cools to room temperature while maintaining a positive pressure inside the powder transfer vessel. Under positive nitrogen pressure of greater than 1.5 psig, the process and resultant powder will remain sterile in the collection vessel with the positive pressure providing a tamper-evident seal for an extended period, e.g., 14-28 days.

In various embodiments, the spray dryer may utilize a drying gas having an inlet temperature of about 150° C.-250° C., and preferably of about 170° C.-230° C. The drying gas may have an outlet temperature of about 70° C.-150° C., and preferably of about 70° C.-120° C., for example the outlet temperature during the production of Azithromycin is 75°. The nitrogen gas flow rate can range from 650-750 kg/hour but other flow rates can be used to accommodate the rate of the feedstock, equipment and temperature variations.

Collection vessel 200 may then be transferred to an aseptic powder filler and used as a direct transfer vessel for the sterile powder to the filler system. A second valve, such as a CHARGEPOINT® Excel valve as described above, may be used to transfer the sterile powder to the filler system. The powder may then be transferred to dosage vials. After filling, stoppering, and crimping the vials, the vials may be inspected, labeled, and boxed for storage until final release is attained.

The sterility of the spray drying system may be maintained using a variety of methods. The system may include filter elements, e.g., removable filter elements, and the system can be cleaned using a fully automated clean in place (CIP) process. After the CIP process is completed, the filter elements can be installed and may also be integrity tested. Upon acceptable filter integrity testing, the system can be steam sterilized using a fully automated steam in place (SIP) process. When steam sterilization is complete, the spray dryer can be purged with sterile filtered nitrogen gas to remove residual steam and to maintain a positive internal pressure.

Pressure within the system can be maintained using sterile filtered nitrogen gas until the spray drying process is complete.

Two mixing systems may be provided so that one system can be used to feed the spray dryer for approximately 18 hours while the second system is cleaned in place, sterilized in place, and used to mix solution that may be spray dried in the following 18 hour period.

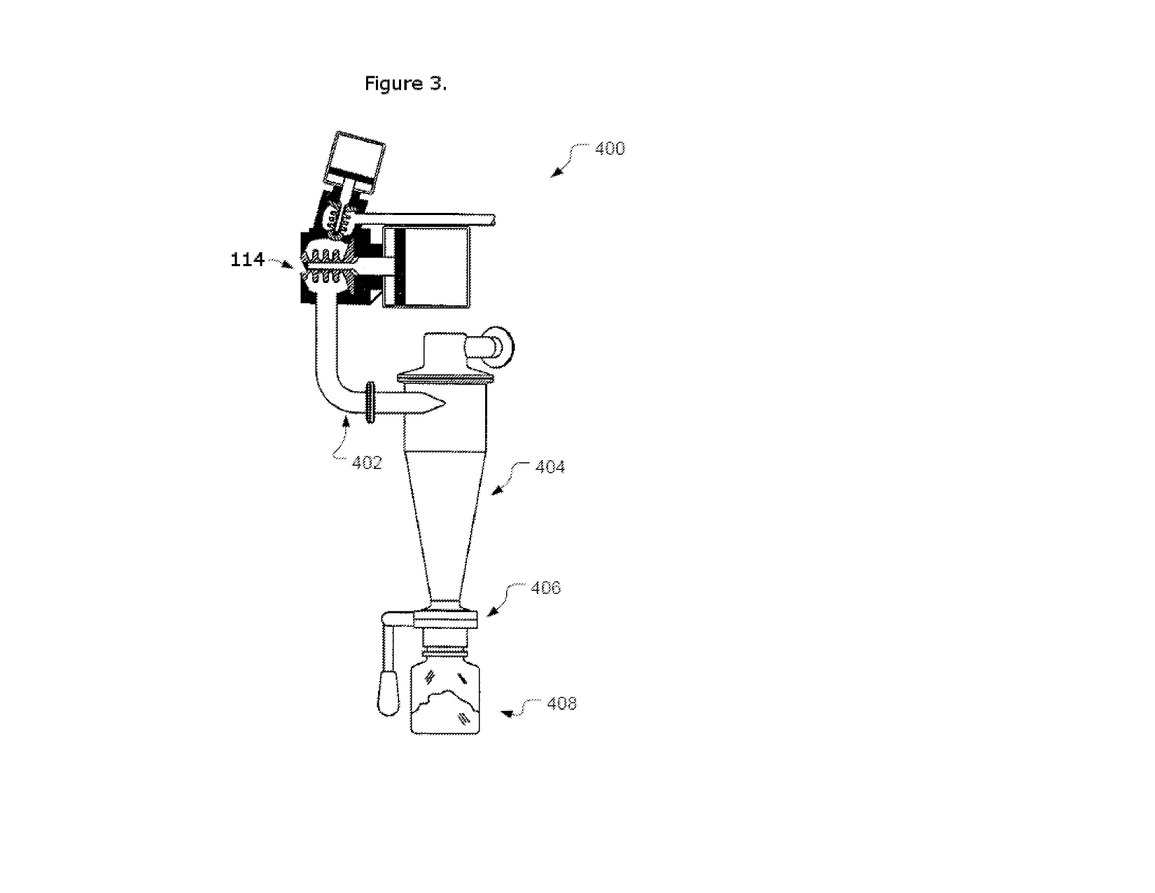

In a further embodiment, the spray drying system of the invention includes a powder sampling system 400, shown in FIG. 3, which takes advantage of the high operating pressure of the system as the motive force to discharge powder from a spray dryer product chute 402, which may be used to convey product from the base of the cyclone of the spray dryer to the collection vessel. A valve 406 allows for the sterile transfer of powder from the drying chamber 114 to a receptacle 408 mounted at the discharge of a lab-type sampler cyclone 404 in communication with the spray dryer product chute 402. Transfer of product from the spray dryer product chute 402 to the sampler cyclone 404 is sterile and the critical integrity of the system is not compromised.

The spray dried vancomycin HCl, daptomycin, azithromycin, cyclophosphamide, or voriconazole of the present application has advantages over lyophilized formulations. For example, the spray drying process of the present application can be used to produce powder more quickly than a standard lyophilization process, with a significant reduction in processing times and operator manipulations/interventions.

In addition, the spray dried vancomycin HCl, daptomycin, azithromycin, cyclophosphamide, or voriconazole formulations allow for reconstitution times at least as fast as the lyophilized formulations. The formulation is suitable for delivery by IV infusion to a patient.

### EXAMPLES

### Example 1

Five batches containing vancomycin HCl (15% by weight), PEG-400 (3% by weight), and mannitol (3% by weight) in WFI were made and then spray dried according the above method. Resultant spray dried powders were tested as shown in Table 3.

TABLE 3

| | | | | Vancomycin B/ |
|---|---|---|---|---|
| | Batch | | Reconstitution | Single Largest |
| Batch # | Size | % Moisture* | Time (seconds) | Impurity (%) |
| 1 | 1 L | 1.7 (GC) | 30 | Not Tested |
| 2 | 5 L | 0.79/0.82 (KF) | 30 | Not Tested |
| 3 | 2 L | 0.77/0.74 (KF) | 23 | 92.5/1.7 |
| 4 | 1 L | 1.0/0.9/1.1 (KF) | 25/27/27/26 | 94.5/1.3 |
| 5 | 10 L | 0.8/0.9/0.8/0.9 (KF) | 31/36/31/32 | 93.9/1.3 |

*GC = Gas chromatography; KF = Karl Fisher

### Example 2

Vancomycin HCl, PEG-400, and mannitol were dissolved at various concentrations in ethanol and WFI. The various formulations were spray dried as described above. Resultant powders were tested as shown in Table 4.

TABLE 4

| | | | | Vancomycin |
|---|---|---|---|---|
| | Formulations | | | B/Single |
| | Vancomycin/PEG- | % | Reconstitution | Largest |
| Batch | 400/Mannitol/ | Moisture | Time | Impurity |
| # | Ethanol/WFI | (KF) | (seconds) | (%) |
| 6 | 15%/3%/3%/10%/69% | 0.8 | 45.4 | 94.2/1.5 |
| 7 | 18%/3%/3%/10%/66% | 1.2 | 47.5 | 94.5/1.5 |
| 8 | 15%/2%/5%/0%/78% | 0.7 | 22.9 | 94.6/1.5 |
| 9 | 20%/3%/3%/15%/59% | 0.7 | 61.6 | 94.6/1.6 |

### Example 3

Spray drying of various formulations of vancomycin HCl was performed using different parameters for the spray drying process, such as gas temperature, nozzle specifications, and utilizing production scale spray drying equipment. The results of the experiments are shown below in Tables 5 and 6.

TABLE 5

| | Test No. | | | | |
|---|---|---|---|---|---|
| | Test 1 | Test 2 | Test 3 | Test 4 | Test 5 |
| Duration, start-stop | 10:00-10:57 | 11:30-12:05 | 13:49-14:47 | 15:27-15:53 | 10:07-10:52 |

Drying gas (Nitrogen)

| | | | | | |
|---|---|---|---|---|---|
| Main Process gas, kg/hr | 1250 | 1250 | 1250 | 1250 | 1250 |
| Inlet Temperature, ° C. | 180 | 180 | 180 | 210 | 215 |
| Outlet Temperature, ° C. | 111 | 97-100 | 88-90 | 100 | 102-103 |

Atomizer

| Specification | Pressure Nozzle | | | | |
|---|---|---|---|---|---|
| Nozzle pressure, bar(g) | 22-26 | 43-47 | 37-42 | 48-53 | 24-25 |
| Nozzle air temp., ° C. | 60 | 60 | 60 | 60 | 60 |
| Nozzle protection gas rate, | 95 | 95 | 95 | 95 | 95 |
| kg/hr | | | | | |
| Nozzle combination | 67/20 | 67/20 | 67/17 | 67/17 | 57/17 |

Feed (Vancomycin (15%)/PEG-400 (3%)/mannitol (3%) WFI (79%))

| | | | | | |
|---|---|---|---|---|---|
| Density, g/ml | 1.05 | 1.05 | 1.05 | 1.05 | 1.05 |
| Feed rate, kg/hr | 28-29 | 43-44 | 53 | 60-61 | 60 |

Powder Analysis

| | | | | | |
|---|---|---|---|---|---|
| Residual moisture % (Karl | 1.14/1.24 | 1.22/1.31 | 2.35/2.62/ | 1.93/ | 1.44/ |
| Fischer) | | | 2.37 | 1.85 | 1.60 |
| Reconstitution time, (seconds) | 20 | 21 | 15 | 21 | 26 |
| Particle size, D50, μm | 32 | 33 | 36 | 32 | 43 |

Yield

| | | | | | |
|---|---|---|---|---|---|
| Collected product, cyclone, kg | 4.5 | 4.6 | 8.9 | 4.9 | 8.1 |
| Collected product, bag filter, | 0.3 | | | | |
| kg | | | | | |

TABLE 6

| | Test No. | | | |
|---|---|---|---|---|
| | Test 6-1 | Test 6-2 | Test 6-3 | Test 6-4 |
| Duration, start:stop | 11:14-12:11 | 12:11-13:15 | 13:15-14:15 | 14:15-15:08 |

Drying gas (Nitrogen)

| | | | | |
|---|---|---|---|---|
| Main Process gas, kg/hr | 1250 | 1250 | 1250 | 1250 |
| Inlet Temperature, ° C. | 215 | 215 | 215 | 215 |
| Outlet Temperature, ° C. | 102-104 | 102-104 | 102-104 | 102-104 |

Atomizer

| Specification | Pressure Nozzle | | | |
|---|---|---|---|---|
| Nozzle pressure, bar(g) | 24-25 | 24-25 | 24-25 | 24-25 |
| Nozzle air temp., ° C. | 60 | 60 | 60 | 60 |
| Nozzle protection gas rate, kg/hr | 95 | 95 | 95 | 95 |
| Nozzle combination | 57/17 | 57/17 | 57/17 | 57/17 |

Feed (Vancomycin (15%)/PEG-400 (3%)/mannitol (3%), WFI (79%))

| | | | | |
|---|---|---|---|---|
| Density, g/ml | 1.05 | 1.05 | 1.05 | 1.05 |
| Feed rate, kg/hr | 60 | 60 | 60 | 60 |

Powder Analysis

| | | | | |
|---|---|---|---|---|
| Sample time | 12:11 | 13:15 | 14:15 | 15:08 |
| Sample point | Cyclone | Cyclone | Cyclone | Cyclone |
| Residual moisture % (Karl Fischer) | 1.81/1.81 | 1.80/1.87 | 1.60/1.72 | 1.69/1.56 |
| Reconstitution time (seconds) | 20 | 23 | 18 | 21 |
| Particle size, D50, μm | 44 | 44 | 47 | 42 |

Yield

| | | | | |
|---|---|---|---|---|
| Collected product, cyclone, kg | 10.3 | 11.8 | 11.2 | 9.3 |
| Collected product, bag filter, kg | 0.3 | | | |

### Example 4

Batches for stability testing were made using the spray dried formulation vancomycin HCl (15% by weight), PEG-400 (3% by weight), mannitol (3% by weight) in WFI, with the API sourced from Abbott Laboratories (North Chicago, Ill.). Spray dried batches were stored at 25° C./60% Relative Humidity (RH), 30° C./60% RH, and 40° C./75% RH for three months. The stability data on moisture by Karl Fischer, pH (of the reconstituted solution), reconstitution time and chromatographic purity are presented in Table 7. The chromatographic purity was examined using the analytical HPLC method described in the current USP (United States Pharmacopeia) monograph for vancomycin. The test results for the API lot used to make the spray dried product were also listed for comparison.

The chromatographic purity of vancomycin B for all samples tested in this study were within USP specifications. All samples were stored at the accelerated conditions of 40° C./75% RH for 3 months and met USP criterion for vancomycin B by chromatographic purity (NLT 80% vancomycin B).

TABLE 7

| | | | Reconstitution | | |
|---|---|---|---|---|---|
| | Stability | | Time(s) in | % | |
| Sample | Temp/Time | Vancomycin B | Min:Sec | Moisture | pH |
| API | Initial | 91.9% | Not performed | Not | Not |
| Lot 09097619 | | | | performed | performed |
| Lot 10-0212 | Initial | 91.7% | 0:59 | 0.9 | 3.3 |
| Lot 10-0212 | 25° C./1 Month | 91.2% | 0:23, 0:23, 0:22 | 1.0 | 3.3 |
| Lot 10-0212 | 30° C./1 Month | 91.2% | Not performed | 1.0 | 3.3 |
| Lot 10-0212 | 40° C./1 Month | 90.8% | 0:21, 0:21, 0:21 | 1.0 | 3.3 |
| Lot 10-0212 | 25° C./2 Month | 90.6% | 0:21, 0:20, 0:20 | 1.1, 1.2 | 3.4 |
| Lot 10-0212 | 30° C./2 Month | 90.2% | 0:30, 0:18, 0:16 | 1.1, 1.2 | 3.3 |
| Lot 10-0212 | 40° C./2 Month | 89.2% | 0:17, 0:20, 0:16 | 1.1, 1.2 | 3.4 |
| Lot 10-0212 | 25° C./3 Month | 90.8% | 0:20 | 1.1 | 3.3 |
| Lot 10-0212 | 30° C./3 Month | 90.6% | 0:21 | 1.1 | 3.4 |
| Lot 10-0212 | 40° C./3 Month | 89.4% | 0:17 | 1.1 | 3.4 |

### Example 5

Three vancomycin formulations were prepared in WFI or citrate buffer. The solutions were spray dried as described above. Resultant powders were tested along with the vancomycin HCl API for impurities, reconstitution time and moisture as shown in Table 8.

TABLE 8

| | | Spray Dry | | |
|---|---|---|---|---|
| | | Formulation | Spray Dry | Spray Dry |
| | | 1 | Formulation 2 | Formulation 3 |
| | | API | | |
| | | | API 15%, | API 15%, |
| | | | PEG-400 2.5%, | PEG-400 |
| | | API15%, | Mannitol 2.5%, | 3.0%, |
| | | WFI | WFI | citrate buffer |
| Vancomycin B | 94.9% | 92.7% | 94.4% | 94.3% |
| N-Demethyl- | 0.6% | 0.7% | 0.6% | 0.6% |
| vancomycin | | | | |
| Other | 4.5% | 6.6% | 5.0% | 5.1% |
| Reconstitution | N/A | 21, 27, 28 | 25, 26, 31 | 69, 47, 40 |
| Time (seconds) | | | | |
| Moisture % | Not | 2.1 | 1.5 | 1.3 |
| n = 2 | tested | | | |

### Example 6

A 1 kilogram batch of daptomycin (10% by weight) with PEG-400 (3% by weight) and mannitol (3% by weight) in WFI was formulated for spray drying. The pH of the final solution was adjusted to 4.7 using 2N sodium hydroxide. Spray drying was accomplished as described above with pilot scale equipment (open system) using 4 different processing schemes as shown in Table 9. Samples from each run were weighed out and reconstituted using an aqueous 0.9% sodium chloride solution. Reconstitution times are provided in Table 9. Results for a sample of CUBICIN® daptomycin for injection (500 mg lyophilized powder per vial, Cubist Pharmaceuticals, Inc.) reconstituted with 10 mL 0.09% sodium chloride are also shown to demonstrate a favorable reconstitution time of the spray dried daptomycin formulations of the invention.

TABLE 9

| | Outlet | Feed | N2 gas | | | | Reconstitution |
|---|---|---|---|---|---|---|---|
| | Temp. | rate | flow | | | Reconstitution | Time |
| Run | (deg C.) | (psi) | (kg/hr) | Nozzle | Weight | volume | (min:sec) |
| 1 | 115 | 95 | 95 | #1 | 402.5 mg | 5 mL | 3:40 |
| 2 | 95 | 95 | 95 | #1 | 801.3 mg | 10 mL | 1:37 |
| 3 | 90 | 100 | 95 | #1 | 800.6 mg | 10 mL | 1:47 |
| 4 | 85 | 100 | 95 | #1 | 800.9 mg | 10 mL | 3:10 |
| Cubicin ® | — | — | — | — | 500 mg | 10 mL | 5:53 |

### Example 7

A 0.4 kilogram batch of daptomycin (12% by weight) with PEG-400 (3% by weight) and mannitol (5% by weight) in WFI was formulated for spray drying. The pH of the final solution was adjusted to 4.9 with 2N sodium hydroxide. Spray drying was done with pilot scale equipment (open system) using the processing scheme shown in Table 10. A sample was weighed out and reconstituted using an aqueous 0.9% sodium chloride solution; the reconstitution time is provided in Table 10. Results for a sample of CUBICIN® daptomycin for injection (500 mg per vial lyophilized powder, Cubist Pharmaceuticals, Inc.) reconstituted with 10 mL 0.09% sodium chloride are also shown to demonstrate a favorable reconstitution time of the spray dried daptomycin formulations of the invention.

TABLE 10

| | Outlet | Feed | N2 gas | | | | Reconstitution |
|---|---|---|---|---|---|---|---|
| | Temp. | rate | flow | | | Reconstitution | Time |
| Run | (deg C.) | (psi) | (kg/hr) | Nozzle | Weight | volume | (min:sec) |
| 1 | 105 | 85 | 100 | #1 | 307.2 mg | 5 mL | 1:06 |
| Cubucin ® | — | — | — | — | 500 mg | 10 mL | 5:53 |

### Example 8

A 2 liter batch of azithromycin (7.2% by weight) and citric acid (5.6% by weight) in WFI was formulated for spray drying. The pH of the solution was adjusted to 6.5 with 10% sodium hydroxide. The 2 liter batch was divided into four 500 mL portions with subsequent addition of excipients to the formulation as shown in Table 11. The four different azithromycin formulations were spray dried with pilot scale equipment (open system) using the following processing conditions: outlet temperature of 75° C., feed rate of 80 psi, N₂ gas flow rate of 80 kg/hr. A sample from each spray dried formulation was weighed and reconstituted in WFI (approximately 1 gram per 10 mL), and the reconstitution time for the sample from each formulation is shown in Table 11.

TABLE 11

| | | Citric | | PEG- | |
|---|---|---|---|---|---|
| | | acid | Mannitol | 400 | |
| Formu- | Azithromycin | (% by | (% by | (% by | Reconstitution |
| lation | (% by weight) | weight) | weight) | weight) | Time (min:sec) |
| 1 | 6.7 | 5.2 | 6.7 | 0 | 0:24 |
| 2 | 7.0 | 5.5 | 2.3 | 2.3 | 0:30 |
| 3 | 7.2 | 5.6 | 0 | 0 | 0:28 |
| 4 | 6.9 | 5.4 | 3.5 | 0 | 0:20 |

### Example 9

Two 500 mL batches of azithromycin (7.2% by weight) and citric acid (5.6% by weight) and mannitol in WFI were formulated for spray drying. The pH of the solution was adjusted to around 6.5 with 10% sodium hydroxide. The formulation specifics for each 500 mL batch are shown in Table 12. The two different azithromycin formulations were spray dried with pilot scale equipment (open system) using the following processing conditions: outlet temperature of 75° C., feed rate of 80 psi, N₂ gas flow rate of 80 kg/hr. A sample from each spray dried formulation was weighed and reconstituted in WFI and the reconstitution time for the sample from each formulation is shown in Table 12.

TABLE 12

| | | Citric | | PEG- | |
|---|---|---|---|---|---|
| | | acid | Mannitol | 400 | Reconstitution |
| Formu- | Azithromycin | (% by | (% by | (% by | Time |
| lation | (% by weight) | weight) | weight) | weight) | (min:sec) |
| 1 | 7.2 | 5.6 | 3.6 | 0 | 0:31 |
| 2 | 7.2 | 5.6 | 1.8 | 0 | 0:43 |

Although various specific embodiments of the present invention have been described herein, it is to be understood that the invention is not limited to those precise embodiments and that various changes or modifications can be affected therein by one skilled in the art without departing from the scope and spirit of the invention

1. A method for preparing a powdered daptomycin, azithromycin, cyclophosphamide, or voriconazole pharmaceutical formulation for injection, the method comprising:
providing a solution comprising daptomycin, azithromycin, cyclophosphamide, or voriconazole and polyethylene glycol (PEG); and
spray drying the solution to form a powder that can be reconstituted and administered by IV infusion.

2. The method of claim 1, wherein the solution further comprises mannitol.

3. The method of claim 2, wherein the solution comprises about 10-20% by weight daptomycin, about 1-10% by weight PEG, and about 2-8% by weight mannitol.

4. The method of claim 2, wherein the solution comprises about 5-10%% by weight azithromycin and about 4-7% citric acid in water.

5. The method of claim 4, wherein the solution comprises about 1-5% PEG and about 1-7% by weight of mannitol.

6. The method of claim 1, wherein the PEG is PEG-400.

7. The method of claim 3, wherein the solution has a pH of about 4.0-5.5.

8. The method of claim 4, wherein the solution has a pH of about 6.0-7.0.

9. The method of claim 1, wherein the spray drying is undertaken in an aseptic environment.

10. The method of claim 1, wherein the spray drying is undertaken at a pressure of 1.5-7.5 psig.

11. The method of claim 12, further comprising transferring the powder under positive pressure to a pressurized vessel.

12. A method for preparing a powdered azithromycin formulation for injection, the method comprising:
providing a solution comprising azithromycin and citric acid; and
spray drying the solution to form a powder that can be reconstituted and administered by IV infusion.

13. The method of claim 12, wherein the solution comprises about 5-10% azithromycin by weight and about 4-8% citric acid by weight.

14. The method of claim 12, wherein the solution further comprises about 2-8% mannitol.

15. The method of claim 12, wherein the solution further comprises about 2-8% PEG-400.

16. A method for preparing a powdered daptomycin formulation for injection, the method comprising:
providing a solution comprising daptomycin and citric acid; and
spray drying the solution to form a powder that can be reconstituted and administered by IV infusion.

17. The method of claim 16, wherein the solution comprises about 5-20% daptomycin, about 1-10% mannitol by weight and about 1-10% PEG-400 by weight.

18. The method of claim 16, wherein the solution comprises about 10-12% daptomycin by weight.

19. The method of claim 18, wherein the solution comprises about 3-5% mannitol by weight.

20. The method of claim 18, wherein the solution comprises about 3% PEG-400 by weight.
